(19) United States
(12) Patent Application Publication
(10) Pub. No.: US 2025/0249163 A1
(43) Pub. Date: Aug. 7, 2025

(54) AUTOMATED MONITORING OF EXTRACORPOREAL SUPPORT PATIENT CARE AND INTERACTIVE USER INTERFACE THEREFOR

(71) Applicant: Inspira-Technologies OXY B.H.N. LTD, Ra'anana (IL)

(72) Inventors: Tomer Carmeli, Alonei Abba (IL); Adi Rizansky Nir, Herzliya (IL)

(21) Appl. No.: 18/434,809

(22) Filed: Feb. 7, 2024

(51) Int. Cl. A61M 1/36

(52) U.S. Cl. CPC A61M 1/3653; A61M 2205/18; A61M 2205/502

(57) ABSTRACT

A method, system, and computer program for automated monitoring of extracorporeal support patient care. An interactive user interface adapted to receive user selection of an operating mode for treatment of a patient supported with an extracorporeal support device is provided. A checklist associated with the operating mode selected is retrieved from a database, the checklist including a plurality of action items required to be performed by a medical staff in treatment of the patient at a set of time intervals. The plurality of action items are presented via the interactive user interface in accordance with the set of time intervals for prompting user confirmation of performing a respective portion of the plurality of action items presented at a respective timepoint. An alert is outputted in response to not receiving user confirmation within a predetermined timeout.

### FIELD OF INVENTION

The present disclosure relate to healthcare management and, more specifically, but not exclusively, to automated monitoring of extracorporeal support patient care.

### BACKGROUND

Extracorporeal support, often referred to as extracorporeal life support (ECLS) or extracorporeal membrane oxygenation (ECMO), is a critical medical intervention employed to sustain patients with life-threatening cardiac or respiratory conditions. This technique involves the temporary diversion of blood from the patient's circulatory system to an external circuit outside the body, which provides oxygenation and circulation support. it is aimed to serve as a bridge to recovery or transplantation, enabling patients' vital organs, particularly the heart and lungs, to rest and heal during the treatment of underlying medical conditions.

Extracorporeal support may be utilized in various clinical scenarios, including for example cases of acute respiratory distress syndrome (ARDS), cardiogenic shock, congenital heart defects, and high-risk cardiac surgeries. Extracorporeal support systems have proven effective in providing life-sustaining support, albeit with certain limitations and inherent risks. The decision to initiate extracorporeal support treatment is made by a multidisciplinary team of medical professionals, considering the patient's overall condition, the underlying cause of their organ failure, and the potential risks and benefits.

Extracorporeal support is a complex and resource-intensive therapy, involving a series of multistep actionable processes and typically requiring intervention or supervision by several healthcare specialists of different expertise, including both physicians and technicians. The process begins with cannulation, namely the insertion of catheters (tubes) into the patient's blood vessels, usually in the neck, groin, or chest. These catheters are connected to the extracorporeal support circuit. There are two primary types of cannulation modes: (i) veno-venous (VV), used primarily for respiratory support, and in which one catheter is placed in a large vein (usually the inferior vena cava via the femoral vein) to withdraw blood from the patient and another catheter is placed in another vein to return oxygenated blood back into the patient's circulatory system (usually the superior vena cava via the right internal jugular vein); and (ii) veno-arterial (VA), which provides both cardiac and respiratory support and is used when the heart's pumping function is compromised, and involves placing one catheter in a large vein for blood withdrawal and another catheter in a large artery (typically the femoral artery) for blood return. Next, an oxygenation and circulation process is initiated, during which the blood withdrawn from the patient's body flows through the extracorporeal support circuit, which consists of a pump and an oxygenator. The blood is pumped through the circuit using a mechanical pump, which replaces the pumping action of the heart, thus ensuring a continuous flow of blood through the circuit. Within the extracorporeal support circuit, the blood passes through an oxygenator, which adds oxygen to the blood and removes carbon dioxide, thus mimicking the gas exchange function of the lungs.

Throughout the extracorporeal support process, the patient's vital signs, oxygen levels, and other important parameters are closely monitored. Healthcare professionals, including perfusionists and extracorporeal support specialists, make adjustments to the extracorporeal support system settings as needed to maintain appropriate blood flow, oxygenation, and circulation. To prevent clotting within the extracorporeal support circuit, anticoagulant medications (blood thinners) are typically administered. Careful monitoring of the patient's coagulation status is essential to prevent bleeding complications. While on extracorporeal support, the patient may receive additional supportive care, such as mechanical ventilation, or medications to support cardiac function or treat underlying conditions.

As the patient's underlying condition improves, healthcare providers assess whether it is safe to gradually reduce extracorporeal support. This process is known as “weaning.” Once the patient's heart and/or lungs are deemed as being able to function adequately on their own, the extracorporeal support circuit is gradually discontinued, and the catheters are removed in a procedure known as “decannulation”.

There are many limitations and drawbacks of extracorporeal support systems as currently available commercially and usage thereof. One prominent shortcoming of extracorporeal support patient care is an elevated risk of infections associated therewith due to the extracorporeal circuit's exposure to the external environment and blood contact. Another acute problem is bleeding risk and/or complications, as anticoagulation treatment (e.g., administration of medications such as blood thinners and/or the like) is a standard requirement to prevent clot formation within the circuit; however, this can increase the risk of bleeding, necessitating close monitoring and management of the patient's coagulation status. Yet another technical challenge of extracorporeal support is its complexity and resource intensiveness, as the process necessitates specialized equipment and a highly skilled medical team, making it time—and cost-demanding and limiting its accessibility in certain healthcare settings.

### SUMMARY OF INVENTION

It is an object of the present disclosure to describe a system and a method for automated monitoring of extracorporeal support patient care.

The foregoing and other objects are achieved by the features of the independent claims. Further implementation forms are apparent from the dependent claims, the description and the figures.

According to an aspect of some embodiments of the disclosed subject matter there is provided a system for automated monitoring of extracorporeal support patient care, comprising: a processing circuitry adapted to execute a code for: providing an interactive user interface adapted to receive user selection of an operating mode for treatment of a patient supported with an extracorporeal support device; retrieving from a database a checklist associated with the operating mode selected, the checklist comprising a plurality of action items required to be performed by a medical staff in treatment of the patient at a set of time intervals; presenting via the interactive user interface the plurality of action items in accordance with the set of time intervals for prompting user confirmation of performing a respective portion of the plurality of action items presented at a respective timepoint; and outputting an alert in response to not receiving user confirmation within a predetermined timeout.

According to another aspect of some embodiments of the disclosed subject matter there is provided a method for automated monitoring of extracorporeal support patient care, comprising: providing an interactive user interface adapted to receive user selection of an operating mode for treatment of a patient supported with an extracorporeal support device; retrieving from a database a checklist associated with the operating mode selected, the checklist comprising a plurality of action items required to be performed by a medical staff in treatment of the patient at a predetermined synchronized manner; presenting via the interactive user interface the plurality of action items in accordance with the predetermined synchronized manner for prompting user confirmation of performing a respective portion of the plurality of action items presented at a respective timepoint; and outputting an alert in response to not receiving user confirmation within a predetermined timeout.

According to yet another aspect of some embodiments of the disclosed subject matter there is provided a non-transitory computer readable medium storing a software program comprising data and computer implementable instructions for carrying out a method for automated monitoring of extracorporeal support patient care, the method comprising: providing an interactive user interface adapted to receive user selection of an operating mode for treatment of a patient supported with an extracorporeal support device; retrieving from a database a checklist associated with the operating mode selected, the checklist comprising a plurality of action items required to be performed by a medical staff in treatment of the patient at a predetermined synchronized manner; presenting via the interactive user interface the plurality of action items in accordance with the predetermined synchronized manner for prompting user confirmation of performing a respective portion of the plurality of action items presented at a respective timepoint; and outputting an alert in response to not receiving user confirmation within a predetermined timeout.

Optionally, the interactive user interface is provided via an integrated man-machine interface of the extracorporeal support device.

Optionally, the checklist comprising at least one subset of the plurality of action items which members thereof are sequentially ordered, wherein the processing circuitry is further adapted to execute a code for presenting members of a respective one of the at least one subset in a respective sequential order.

Optionally, presenting a subsequent action item of a respective one of the at least one subset is contingent on receiving user confirmation of performing an action item presented.

Optionally, at least one of said plurality of action items is associated with a timer, wherein the processing circuitry is further adapted to execute a code for suspending of prompting user confirmation until expiry of the timer of the respective action item.

Optionally, in response to expiry of the timer a user notification is outputted.

Optionally, an indication of user progress on the checklist is provided via the interactive user interface.

More optionally, the indication of user progress is overlaid on an output provided via another user interface of the extracorporeal support device.

Optionally, the operating mode is selected from the group consisting of: medical staff shift change, supported patient transportation, patient support initiation, and supported patient weaning.

Optionally, information relating to user execution of the checklist is recorded in at least one data store.

More optionally, the at least one data store comprising an electronic medical record.

Optionally, the checklist is loaded from a user input received.

More optionally, the user input is provided in a tabular data format.

Optionally, the operating mode having a mode transition checklist associated therewith, the mode transition checklist comprising another plurality of action items on which switching to the operating mode is preconditioned, wherein the processing circuitry is further adapted to execute a code for: retrieving from the database the mode transition checklist, presenting via the interactive user interface the another plurality of action items for prompting user confirmation of performing thereof, and outputting an alert in response to not receiving user confirmation within a predetermined timeout.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

Unless otherwise defined, all technical and/or scientific terms used herein have the same meaning as commonly understood by one of ordinary skill in the art to which embodiments.

Although methods and materials similar or equivalent to those described herein can be used in the practice or testing of embodiments, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments are herein described, by way of example only, with reference to the accompanying drawings. With specific reference now to the drawings in detail, it is stressed that the particulars shown are by way of example and for purposes of illustrative discussion of embodiments. In this regard, the description taken with the drawings makes apparent to those skilled in the art how embodiments may be practiced. In the drawings:

FIG. 1 is a flowchart schematically representing an optional flow of operations for automated monitoring of extracorporeal support patient care, according to some embodiments;

FIG. 2 is a block diagram of an exemplary apparatus for automated monitoring of extracorporeal support patient care, according to some embodiments;

FIG. 3 is a sequence diagram of various exemplary operational modes of automated monitoring of extracorporeal support patient care, according to some embodiments; and

FIG. 4A, FIG. 4B, FIG. 4C, FIG. 4D, FIG. 4E, FIG. 4F, FIG. 4G, FIG. 4H and FIG. 4I are illustrative screen shots of an exemplary interactive graphical user interface for automated monitoring of extracorporeal support patient care, according to some embodiments.

### DETAILED DESCRIPTION

Some embodiments described in the present disclosure relate to healthcare management and, more specifically, but not exclusively, to automated monitoring of extracorporeal support patient care.

One technical challenge of extracorporeal support patient care is to ensure that standard operating procedures, such as routine and/or exceptional interventions by medical staff (physicians, technicians, and/or the like), are adequately complied with, performed in a timely manner, and/or properly documented for future review.

Upon initiation of extracorporeal support therapy, as well throughout the treatment cycle, there may be required to be carried out several multi-step operational processes which involve a series of actions taken by a medical staff in several timepoints, until the process may be completed. Exemplary scenarios giving rise to processes of this sort may include, but not limited to, weaning a patient from extracorporeal support, transporting a patient receiving extracorporeal support, e.g., for undergoing examination and/or treatment at a different medical facility and/or different location within a same facility.

During such processes, the medical staff may not perform a certain task required, or perform it in a later point in time than a time originally prescribed therefor. Such occurrences may typically result from patient overload, tiredness and/or exhaustion of the medical team, mishandling and/or misinterpretation of information exchanged during staff shift change, and/or the like. When any component of a multi-step actionable process, which may be essential and/or critical for proper patient care, may thus as a result be skipped, deferred, and/or left undone, it can potentially jeopardize patients' lives or, at the very least, extend their hospitalization period.

Under pre-existing approaches, performance of those processes may be monitored manually using a checklist which may be put on paper and/or on a mobile computer (e.g., a tablet, laptop, and/or the like), which are required to be available for the process. Such manual monitoring may thus be overly burdensome on the medical staff, as requiring high level of discipline and consciousness from monitoring personnel, as well as for introducing additional items in an already overcrowded environment, in which such processes may be initiated in an emergency, thus possibly resulting in these items being unavailable at relevant times.

Furthermore, these checklists may typically have variants between institutions and/or settings (e.g., on-site, off-site, and/or the like), based on best practice guidelines of those institutions and/or tailored to their specific clinical conduct an/or the like.

In some embodiments, there is provided an interactive user interface adapted to enable a user, e.g., a medical staff member, a technician, and/or any likewise healthcare professional operating an extracorporeal support device, to activate a module aimed at guiding and accompanying a multi-step actionable process initiated by the user in relation with treatment of a patient using the extracorporeal support device, with timely check items and notifications, such as alerts, reminders, and/or the like.

In some embodiments, the interactive user interface may provide for selection between different operational modes by the user, e.g., according to the type of multistep process to be performed and/or initiated. In response to receipt of the operation mode selection, a checklist associated with the selected operation mode may be obtained, e.g., retrieved from a database, provided as input, and/or the like. The checklist may comprise a plurality of action items required to be performed by medical staff in treatment of the patient and/or in connection with the operation mode selection, optionally at a set of predetermined time intervals (e.g., zero or more times, as may be applicable). The plurality of action items may be presented via the interactive user interface, optionally according to the set of predetermined time intervals, if and/or where applicable, for prompting user confirmation of performing a respective action item and/or combination of action items, as presented at a time. In a case where the user confirmation is not received within a predetermined timeout, an alert may be issued accordingly.

In some embodiments, the interactive user interface may provide for timers adapted to measure an elapsed time interval and/or count down from a predetermined time interval. A timer may be presented via the interactive user interface in association with a respective checklist item and/or items group. The timer may be utilized by the user for providing reminder to move on to a next checklist item upon and/or before expiration of the time interval that has been set, e.g., 5, 10, 15, 30, and/or 45 minutes, 1, 2, 6, and/or 12 hours, 1 day, and/or the like. For example, upon expiry, an indication aimed at drawing user attention to the checklist may be outputted. The indication may be provided, for example, by way of presentation via the interactive user interface, issuance of user notification to a user's hand-held and/or wearable device, such as a mobile phone or a computerized wristwatch, and/or via any likewise communication channels or combinations thereof. Optionally, in response to the user setting the timer, the operation of prompting user confirmation with regard to performance of presented action item(s) may be suspended until expiry of the timer.

In some embodiments, the operating modes and corresponding multistep actionable processes provided for selection by the user may include, for example, initiation of a patient on the extracorporeal support device, transportation of a patient supported by the extracorporeal support device (e.g., to a remote location and/or external facility), weaning of a supported patient from the extracorporeal support device, shift change of medical staff member(s), technician(s), and/or other likewise healthcare personnel, and/or the like.

In some embodiments, the action items in the checklist or a subset of items may be ordered in a sequence and the interactive user interface may be adapted to present the items or subset of items in sequential order accordingly from first to last in the sequence. Optionally, the interactive user interface may not proceed to presenting a next item and/or group of items in the sequence, until user confirmation of performance of a currently presented item is received.

In some embodiments, the interactive user interface may provide an indication of user progress on the checklist, such as for example, a number of checked items out of a total number of items on the checklist, a percentage of checked items out of the total items on the checklist, and/or the like. Optionally, the indication may be provided in a form of a progress bar and/or the like. The indication of progress may be available also if a user exits the checklist module, for example, via a fixed location on a display and/or a repeated time slot on an aural communication, an overlay over an output of the extracorporeal support device, e.g., another user interface thereof, and/or the like.

In some embodiments, a data reader adapted to receive input from a user and/or a computing device coupled to and/or in communication with the extracorporeal support device may be provided. The data reader may be adapted to provide a function of loading a checklist from an input provided, thereby enabling customization and tailoring of the checklist to particular preferences and/or specific needs of users and/or institutions. Optionally, the input of the checklist may be formatted in tabular form, for example, the input may be a spreadsheet file such as Excel available from Microsoft, a tabular text document such as comma separated values (CSV) file, and/or any likewise formats as may be commercially available and/or specially designed. The file may include a list of “check” items, and optionally time intervals specified for respective timers, if and/or where applicable. Optionally, the loading function of such checklists may be subject to access control, e.g., restricted to an administrator user having appropriate permissions and/or credentials on behalf of the institution in question. For example, the loading function may be password protected and/or the like. The checklist may be loaded into the interactive user interface directly, and/or stored at the database for future retrieval. Once loaded, users may have an option to launch a pre-loaded checklist via the interactive user interface, to guide them through the steps of the desired process.

In some embodiments, the timing of checklist initiation and/or completion may be registered in a system log, thereby allowing users to retrospectively learn when meaningful events have taken place, e.g., when looking back at a shift change report, issued by a report generator of the system log. Additionally, or alternatively, the content of the checklists, including the timing of user interactions (checking items) may be communicated to an electronic medical record (EMR) and/or likewise data store, optionally together with patient's vital signs, test results, parameters of the extracorporeal support device (e.g., pump pressure values and/or the like), and/or any likewise relevant data and/or information of interest.

In some embodiments, an operating mode of the interactive user interface may be associated with more than one checklist, for example, one of the checklists may be a precondition for transitioning into that operating mode and another one of the checklists may be a regular checklist performed during that operating mode after transition into it had been completed. The interactive user interface may be adapted, in response to user selection of switching into that operating mode from a different mode, to retrieve the mode transition checklist from the database and present the action items thereon for prompting user confirmation similarly as with other checklists as described herein, where in case the user confirmation not received within a predetermined timeout, an alert may be outputted. Once the mode transition checklist is complete, the interactive user interface may proceed to retrieve from the database and present the regular checklist, optionally according to predetermined time intervals specified therefor.

In some embodiments, the interactive user interface may be provided via an integrated man-machine interface of the extracorporeal support device, such as for example, a built-in display, audio output devices (e.g., speakers), and/or the like. Reminders, alerts, and/or the like may be provided for example in a form of audio and/or visual textual messages, graphical data, lights flickering, sound alarms, and/or any likewise communication forms and/or combinations thereof. Optionally, the interactive user interface may allow users to utilize a pre-embedded institutional (e.g., hospital—and/or likewise facility-prescribed) “alerting program” or likewise scheme detailing how and to whom and/or where alerts may be issued. Additionally or alternatively, users may be able to utilize an available “alerting program” offered as a default program. Optionally, an alerting program may include triggering of one or more physical safety measures and/or contingencies, such as for example, automatic overall facility lockdown for preventing exit and/or entrance, launching of drones with emergency aids, and/or the like.

One technical effect of utilizing the disclosed subject matter is to provide for synchronization of reminders and/or alerts with multistep actionable processes done routinely in the treatment of a patient supported with extracorporeal support. Such alignment and synchronization of reminders and alerts with established multistep processes of a medical team, involving the treatment of a patient receiving extracorporeal support, allows for streamlining performance and enhancing efficiency of medical staff and healthcare professionals, consequently enhancing patient safety as a result.

Moreover, by ensuring proper and timely execution of those multistep actionable processes, a total duration of extracorporeal support therapy may be minimized, thus freeing up extracorporeal support devices for usage in treatment of other patients in due course and/or sooner, resulting in optimal utilization and/or potential increase in overall capacity of such resources, which may be crucial in times of short supply such as during pandemic outbreaks and/or the like.

Another technical effect of utilizing the disclosed subject matter is to provide for user experience and interaction seamlessly intertwined with the multistep actionable processes of extracorporeal support patient care, and optionally, made available via a man-machine interface integrated into the extracorporeal support device itself. Having such checklists on the actual support device can eliminate the need for additional items in an already overcrowded environment, on one hand, yet ensure availability thereof at a time of need, on the other hand.

The disclosed subject matter improves upon and provides many benefits and advantages over pre-existing technologies of extracorporeal support patient care, which do not provide for real time reminders and/or alerts for multistep actionable processes undertaken by the medical staff. Synchronization of alerts and/or notifications in a dedicated, customizable module, may provide for one or more of the following functionalities: (i) drawing attention of medical staff member(s) in real time to an action required to be taken thereby in a timely fashion; (ii) prevent and/or minimize in real time risk of human errors and/or adverse effects to patient's health and/or equipment integrity due to faulty, sub-par, and/or untimely execution; (iii) enable a retrospective assessment of the conduct of the medical staff in response to feedback (e.g., alerts, patient vital signs, device parameters, performance analytics, and/or the like) received in real time.

Other and/or additional technical challenges, approaches, and/or effects improved upon over pre-existing technologies by the disclosed subject matter will be apparent to a skilled artisan in view of the detailed disclosure herein.

Before explaining at least one embodiment in detail, it is to be understood that embodiments are not necessarily limited in its application to the details of construction and the arrangement of the components and/or methods set forth in the following description and/or illustrated in the drawings and/or the Examples. Implementations described herein are capable of other embodiments or of being practiced or carried out in various ways.

Embodiments may be a system, a method, and/or a computer program product. The computer program product may include a computer readable storage medium (or media) having computer readable program instructions thereon for causing a processor to carry out aspects of the embodiments.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random-access memory (RAM), a read-only memory (ROM), an erasable programmable read-only memory (EPROM or Flash memory), a static random-access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of embodiments may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of embodiments.

Aspects of embodiments are described herein with reference to flowchart illustrations and/or block diagrams of methods, apparatus (systems), and computer program products according to embodiments. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

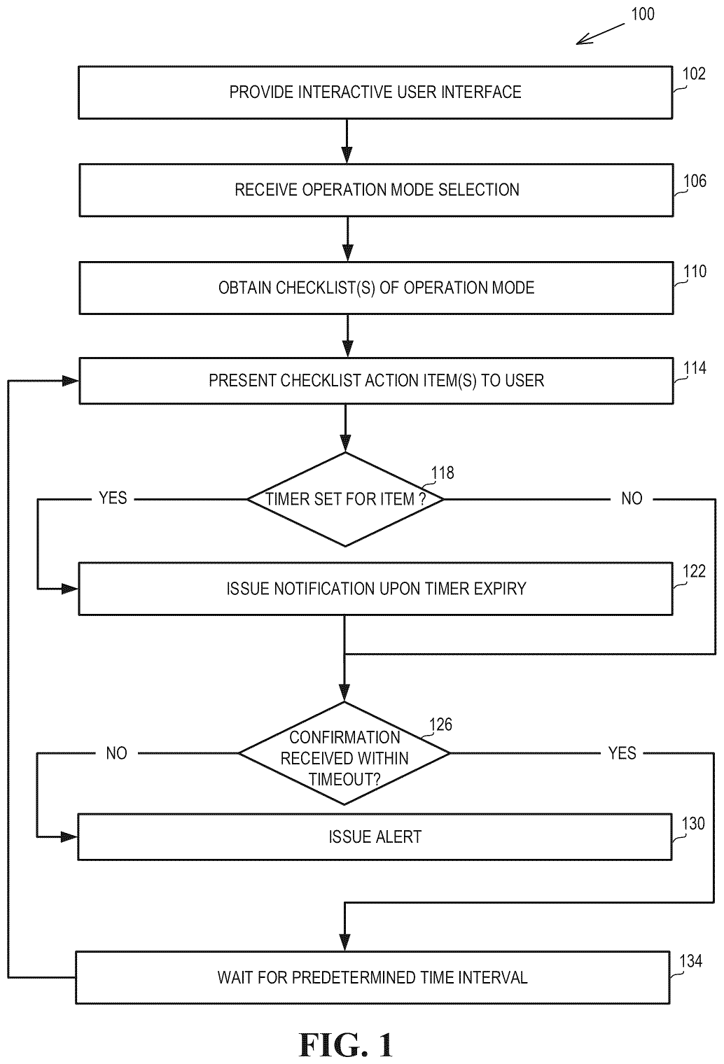

Reference is now made to FIG. 1 which is a flowchart schematically representing an optional flow of operations for automated monitoring of extracorporeal support patient care, according to some embodiments.

At 102 an interactive user interface may be provided. Optionally the interactive user interface may be provided via a man-machine interface integrated into an extracorporeal support device by which a patient is or about to be supported.

At 106 an operation mode selection may be received. The selection may be inputted by a user via the interactive user interface provided at 102.

At 110 one or more checklists associated with the operation mode selected may be obtained. The checklist(s) of the operation mode may be retrieved from a database, loaded from an input received from a user, and/or the like. The checklist(s) may be default checklist(s) preprogrammed for that operation mode, customized checklist(s) preloaded into the database and/or the interactive user interface by an authorized user, and/or the like. The checklist may comprise a plurality of action items to be performed by medical team members and/or healthcare professionals in treatment of the patient using the extracorporeal support device.

At 114 items of a respective checklist as obtained at 110 may be presented via the interactive user interface as provided at 102. An item and/or group of items may be presented to a user for prompting confirmation that the presented item(s) have been performed.

At 118 a check may be made whether an item presented at 114 has a timer associated therewith and if so, whether the timer has been set (i.e., activated) by the user, and accordingly, at 122 a notification may be issued to the user upon expiration of the timer. Optionally the prompting of user confirmation may be suspended until expiration of the timer in question.

At 126 a check may be made whether user confirmation has been received within a timeout period specified, and if not, an alert may be issued accordingly at 130.

At 134 in case repetition of the multistep process accompanied by the checklist(s) obtained at 110 may be required, a waiting period for a predetermined time interval may be instituted, following which the procedure 100 may be resumed at 114.

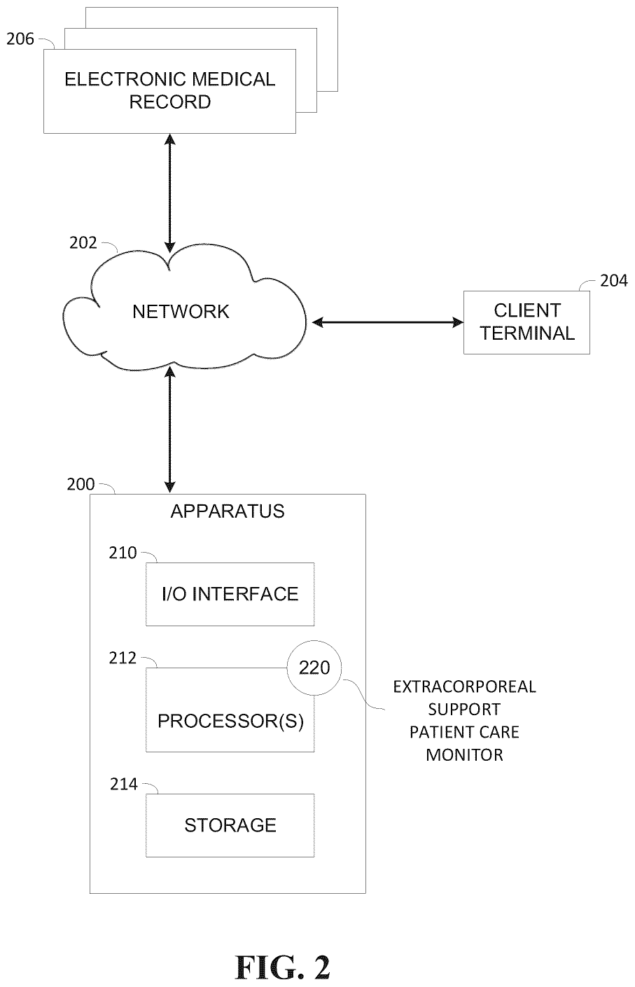

Reference is now made to FIG. 2 which is a block diagram of an exemplary apparatus for automated monitoring of extracorporeal support patient care, according to some embodiments. An exemplary apparatus 200 may be used for performing one or more of the acts for automated monitoring of extracorporeal support patient care such as described with reference to FIGS. 1 and/or 3 herein.

The apparatus 200 may comprise and/or be implemented as, for example, a computer, a server, a computing node, a cluster of computing nodes and/or the like, which may include an Input/Output (I/O) interface 210 for connecting to one or more external devices, systems, services and/or the like, one or more processor(s) 212 for executing a process such as the optional flow of operations 100 as illustrated in FIG. 1, a storage 214 for storing data and/or code (program store), and/or the like.

The I/O interface 210 may include one or more wired and/or wireless network interfaces for connecting to a network 202 comprising one or more wired and/or wireless networks, for example, a Local Area Network (LAN), a Wide Area Network (WAN), a Metropolitan Area Network (MAN), a cellular network, the internet and/or the like. Using the network interface(s) the optimization system 200 may communicate, optionally via the network 202, with one or more (optionally remote, e.g., networked) data resources such as electronic medical records (EMRs) 206 and/or likewise information base(s), which may optionally comprise and/or be implemented as, for example, a server, a computing node, a storage server, a networked database, a cloud service and/or the like. Through the network 202 and/or otherwise, the apparatus 200 may optionally further communicate with one or more client terminals 204, for example, a computer, a server, a laptop, a mobile device, a wearable computer and/or the like used by one or more users, for example, a medical staff member, a technician, a healthcare professional, an administrator, a chief medical officer, and/or the like. Optionally one or more of the client terminals 204 and/or electronic medical records 206 may reside at and/or be coupled to the apparatus 200 locally.

The I/O interface 210 may further include one or more wired and/or wireless I/O interfaces, ports, interconnections and/or the like for connecting to one or more external devices, for example, a Universal Serial Bus (USB) interface, a serial interface, a Radio Frequency (RF) interface, a Bluetooth interface and/or the like. Through the I/O interface 210, the apparatus 200 may communicate with one or more external devices (not shown) attached to the I/O interface(s), for example, an attachable mass storage device, an external media device and/or the like.

The apparatus 200, communicating with one or more of the external devices, client terminals 204, and/or electronic medical records 206, may therefore receive, fetch, collect and/or otherwise obtain data and information required for automated monitoring of extracorporeal support patient care. Such data and information may include, for example: customized checklists for various operation modes; customized alerting programs; user input and/or interaction relating to checklist items, e.g., operating mode selection, performance confirmation of presented item(s), timer setting and/or pausing, and/or the like. Optionally the apparatus 200 may communicate with one or more of the external devices and/or client terminals 204 to output data and information to a user pertaining to automatically monitored extracorporeal support patient care, such as for example, presenting operating modes for user selection, presenting checklist action items for prompting user confirmation of performance thereof, outputting alerts, reminders, notifications etc., and/or the like.

The processor(s) 212, homogenous or heterogeneous, may include one or more processing nodes arranged for parallel processing, as clusters and/or as one or more multi core processor(s). The storage 214 may include one or more tangible, non-transitory persistent storage devices, for example, a hard drive, a Flash array and/or the like. The storage 214 may also include one or more volatile devices, for example, a Random Access Memory (RAM) component, a cache and/or the like. The storage 214 may further comprise one or more local and/or remote network storage resources, for example, a storage server, a Network Attached Storage (NAS), a network drive, a cloud storage service and/or the like accessible via the I/O interface 210.

The processor(s) 212 may execute one or more software modules such as, for example, a process, a script, an application, an agent, a utility, a tool, an Operating System (OS) and/or the like each comprising a plurality of program instructions stored in a non-transitory medium (program store) such as the storage 214 and executed by one or more processors such as the processor(s) 212. The processor(s) 212 may further include, utilize and/or otherwise facilitate one or more hardware modules (elements), for example, a circuit, a component, an integrated circuit (IC), an Application Specific Integrated Circuit (ASIC), a Field Programmable Gate Array (FPGA), a Digital Signals Processor (DSP), a Graphic Processing Units (GPU), an Artificial Intelligence (AI) accelerator and/or the like.

The processor(s) 212 may therefore execute one or more functional modules utilized by one or more software modules, one or more of the hardware modules and/or a combination thereof. For example, the processor(s) 212 may execute an extracorporeal support patient care monitor functional module 220 for providing an interactive user interface via which various multistep actionable processes required to be performed by a medical team in treatment of a supported patient may be guided and accompanied with synchronized alerts, reminders, notifications, and/or the like, and/or performing any likewise automated healthcare monitoring functions, in accordance with some embodiments.

Optionally, the apparatus 200, specifically the extracorporeal support patient care monitor 220 may be adapted to utilize and/or be utilized by one or more cloud computing services, platforms and/or infrastructures such as, for example, Infrastructure as a Service (IaaS), Platform as a Service (PaaS), Software as a Service (Saas) and/or the like provided by one or more vendors, for example, Google Cloud, Microsoft Azure, Amazon Web Service (AWS) and Elastic Compute Cloud (EC2) and/or the like.

One or more of the client terminals 204 may execute one or more applications, services and/or tools for communicating with the apparatus 200 and more specifically with the extracorporeal support patient care monitor 220 to enable one or more of the users to interact with the extracorporeal support patient care monitor 220 and/or functionalities thereof. For example, one or more client terminals 204 may execute a web browser for communicating with the extracorporeal support patient care monitor 220 and presenting a User Interface (UI), specifically a Graphical UI (GUI) which may be used by the respective users to interact with the extracorporeal support patient care monitor 220. In another example, one or more client terminals 204 may execute a local agent which communicates with the extracorporeal support patient care monitor 220 and presents a GUI which may be used by the respective users to interact with the extracorporeal support patient care monitor 220.

The extracorporeal support patient care monitor 220 may be adapted to provide an interactive user interface for guiding and accompanying multistep actionable processes to be performed by medical teams and/or healthcare professionals in treatment of supported patients with synchronized alerts, reminders, notifications and/or the like. The extracorporeal support patient care monitor 220 may be adapted to receive an operating mode selection from a user and retrieve from a database, e.g., retained in the storage 214 and/or the like, a checklist associated with the operating mode selected and comprising a plurality of action items to be performed in treatment of a supported patient. The extracorporeal support patient care monitor 220 may be adapted to present the plurality of action items to the user for prompting confirmation of performance of the action item(s) presented at a time. The extracorporeal support patient care monitor 220 may be adapted to output an alert in case user confirmation is not received within a predetermined timeframe specified.

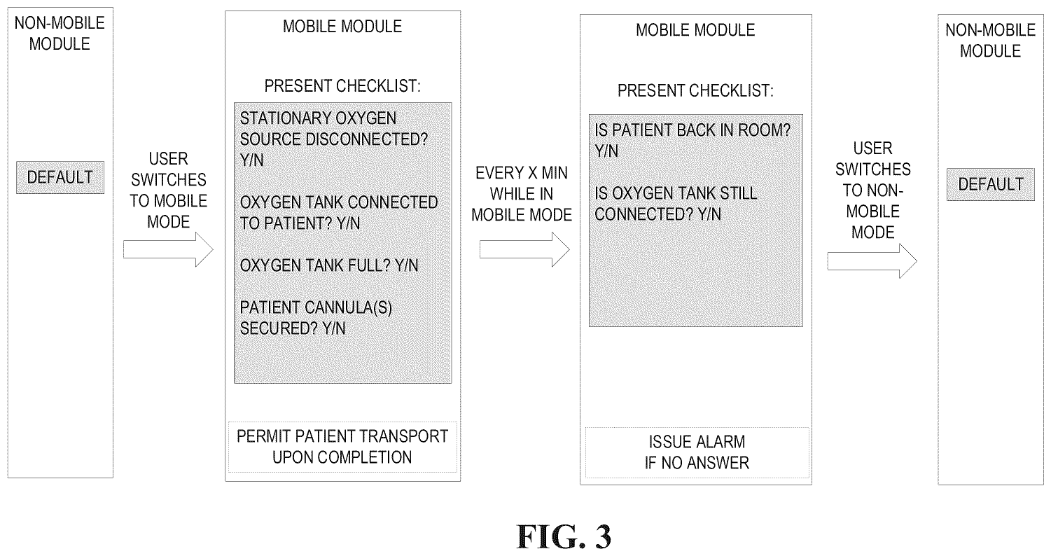

Reference is now made to FIG. 3 which is a sequence diagram of various exemplary operational modes of automated monitoring of extracorporeal support patient care, according to some embodiments.

As shown on FIG. 3, an illustrative example of different operational modes in caring for an extracorporeal supported patient may be in a practical scenario of patient transportation, involving two modes of operation and switching therebetween, a first of which being a mode of non-mobility, considered as a default mode, in which the patient may be disposed at a confined location and connected to fixed immobile equipment and/or infrastructure, e.g., intra-wall oxygen supply and/or the like, and a second of which being a mode of mobility, in which the patient may be transported between an origin and destination locations and/or back to the origin, and during that time be connected to portable equipment and/or vital care supplies, such as for example a mobile oxygen tank and/or the like.

Thus, whenever a patient may be required to be transported while connected to an extracorporeal support device, a staff member responsible for the patient transported may need to disconnect the extracorporeal support device from a fixed oxygen source, e.g., in a wall, and reconnect the extracorporeal support device with an oxygen tank, to be taken with the patient during transport. Similarly, when the patient had been returned into their room, a staff member responsible for the patient may need to reconnect the patient to the fixed oxygen source in the wall.

Accordingly, there may be a need to ensure that staff members transporting the supported patient perform all required and necessary operations prior to, during, and/or following transportation, for example, check that there is enough oxygen in the oxygen tank used in transportation, reconnect the extracorporeal support device to the oxygen source in the wall after the patient had been returned to their room, and/or the like.

As shown on FIG. 3, in some embodiments, in response to a user command to switch from the non-mobility mode to the mobility mode, there may be presented to the user a checklist associated with the mobility mode, which may be a mode transition checklist. As illustrated on FIG. 3, the mode transition checklist presented may comprise a plurality of action items required to be performed by medical staff members as precondition to switching into the operation mode in question. For example, in the illustrative exemplary scenario of patient transportation as described herein, such action items may include a check if a stationary oxygen source is disconnected, a check if an oxygen tank is connected to the patient transported, a check if the oxygen tank is full, a check if cannula(s) connections between the extracorporeal support device and the patient are secured, and/or the like. The mode transition checklist may be presented to the user for soliciting confirmation of performing the presented checklist items, in accordance with some embodiments as described herein. If user confirmation is not received within a predetermined timeout, an alert may be issued accordingly. Otherwise, if the mode transition checklist is completed successfully, switching to the mobility mode may be permitted accordingly.

As shown on FIG. 3, in some embodiments, during operation in the mobility mode, a checklist associated with the mobility mode may be presented to the user, optionally at a set of predetermined time intervals. For example, as illustrated on FIG. 3, the checklist may be presented periodically, e.g., every X minutes for some value of X specified, and/or the like. In some embodiments, the time intervals in the set may not necessarily be regularly spaced, for example, the intervals may be a series of decreasing and/or increasing values, e.g., the checklist may be presented more and more frequently as a total time elapsed since the mobility mode has been switched to grows longer, and/or the like.

As shown on FIG. 3, in some embodiments, in response to user command to switch from the mobility mode to the non-mobility mode, presentation of the checklist of the mobility mode may be terminated and a default functionality may be resumed. Optionally, a (reverse) mode transition checklist required to be completed as precondition for switching back from the mobility mode to the non-mobility mode may be presented, similarly as with relation to the opposite switch, as described herein. For example, the reverse mode transition checklist may include action items such as a check if the stationary oxygen source is connected, a check if the oxygen tank is disconnected, a check if the cannula(s) connections are secured, and/or the like.

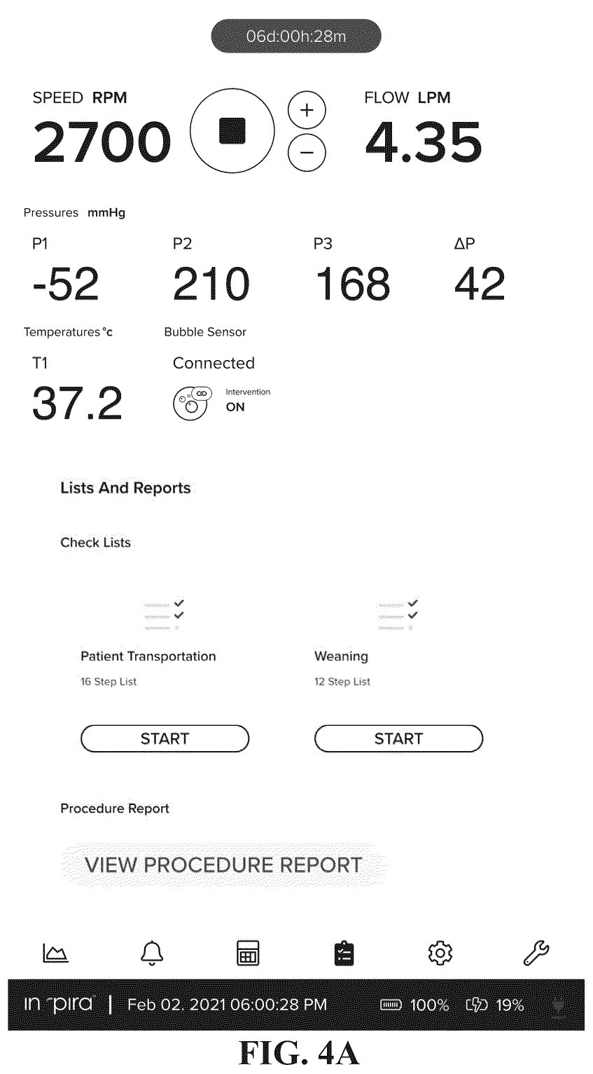

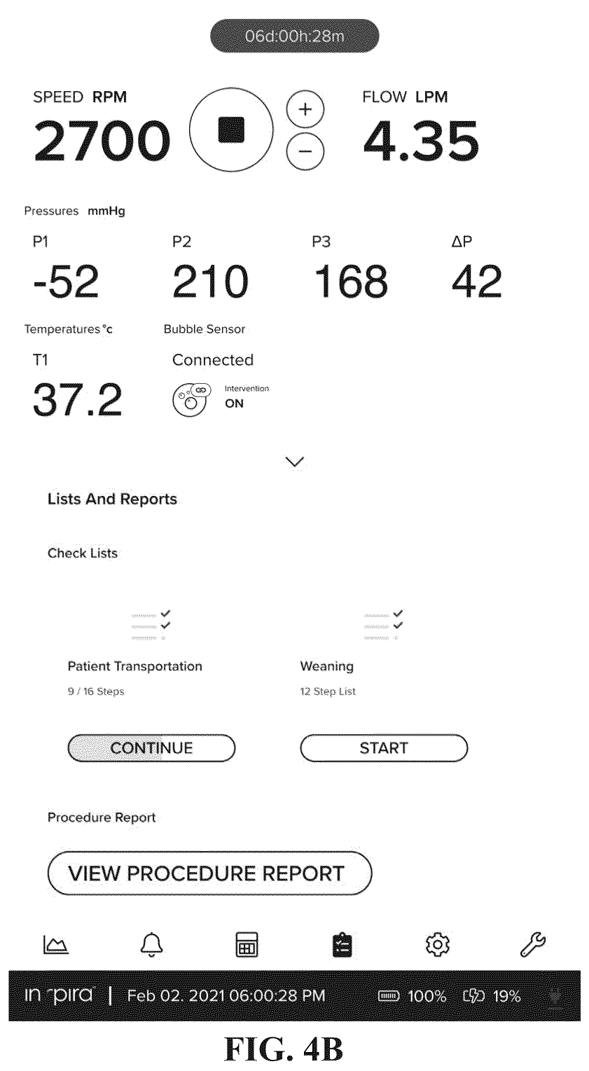

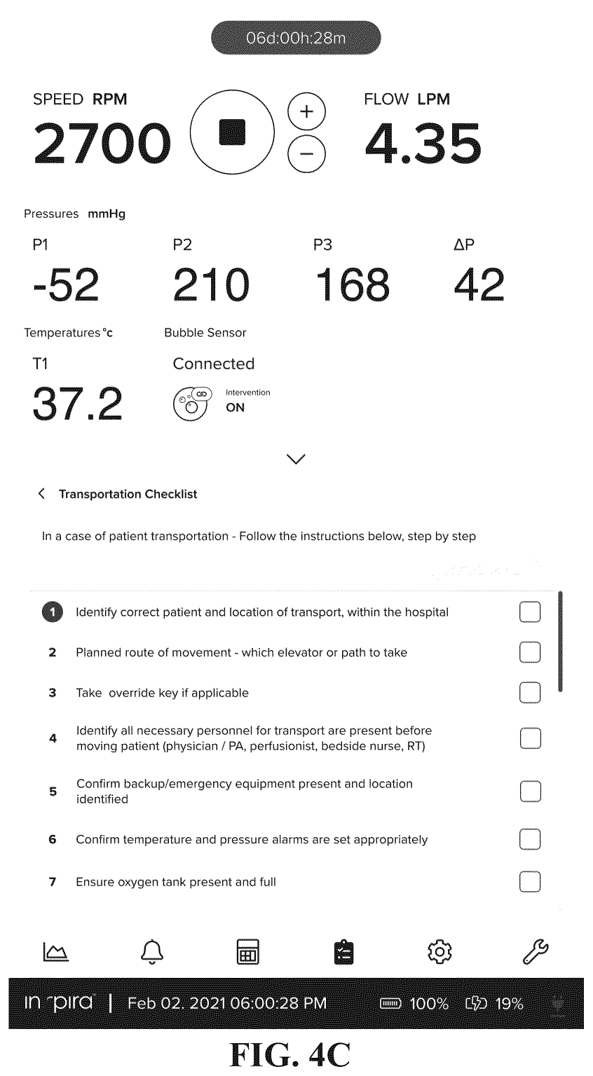

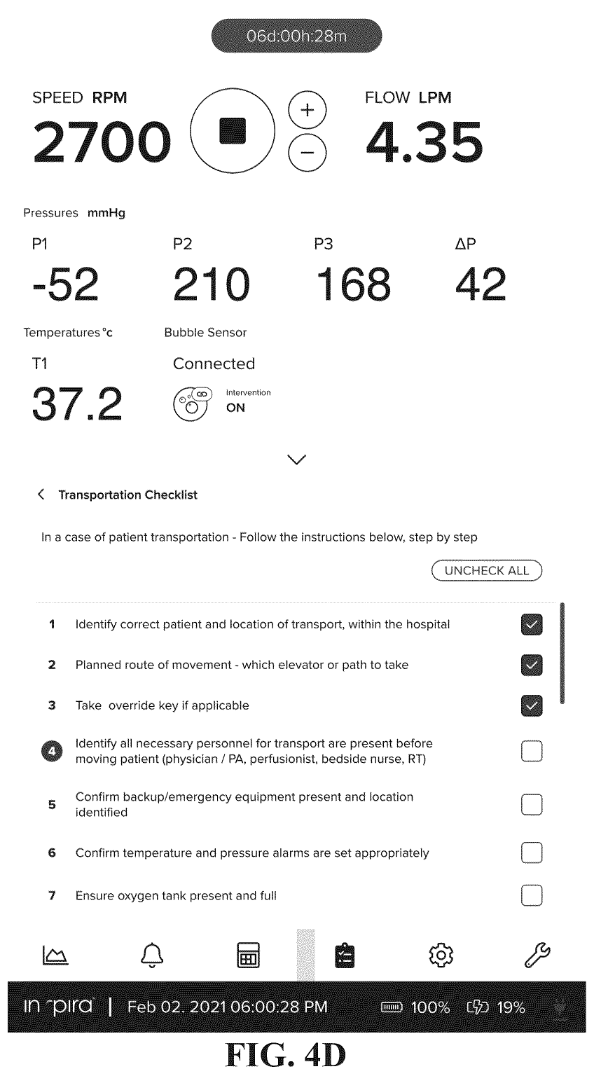

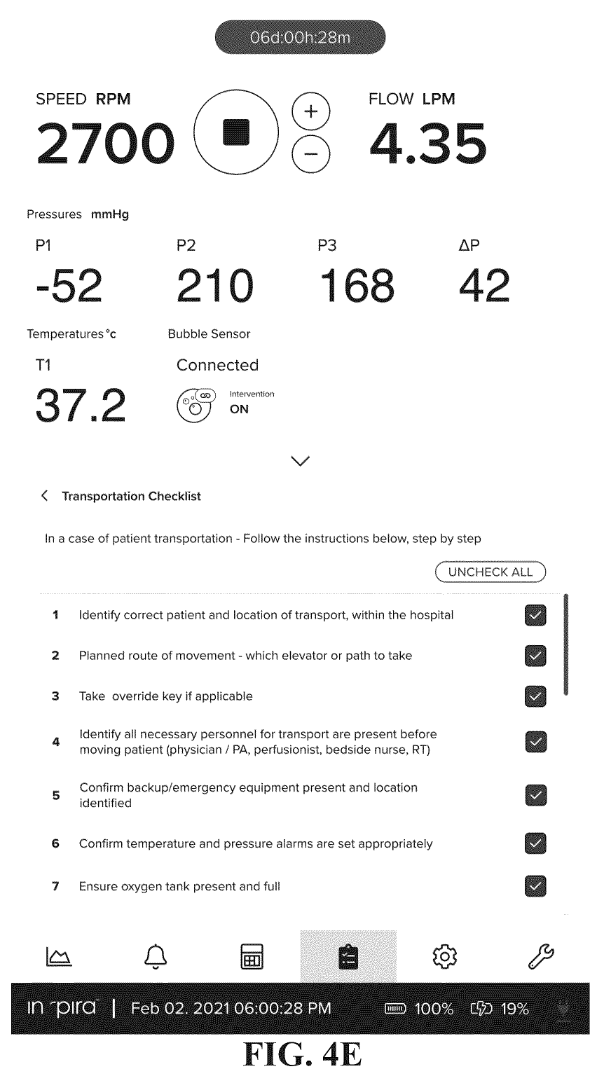

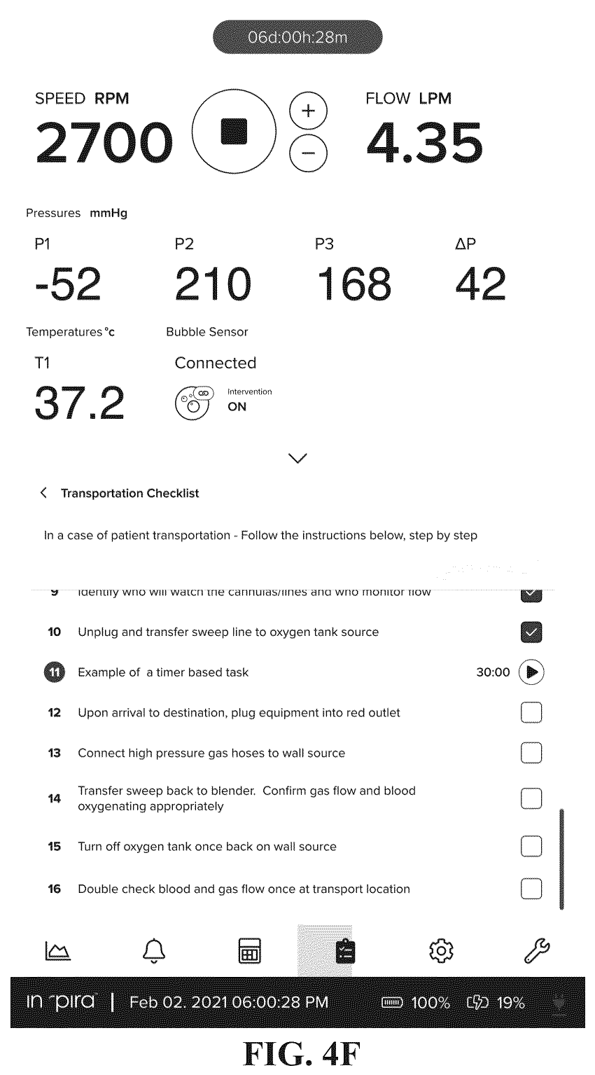

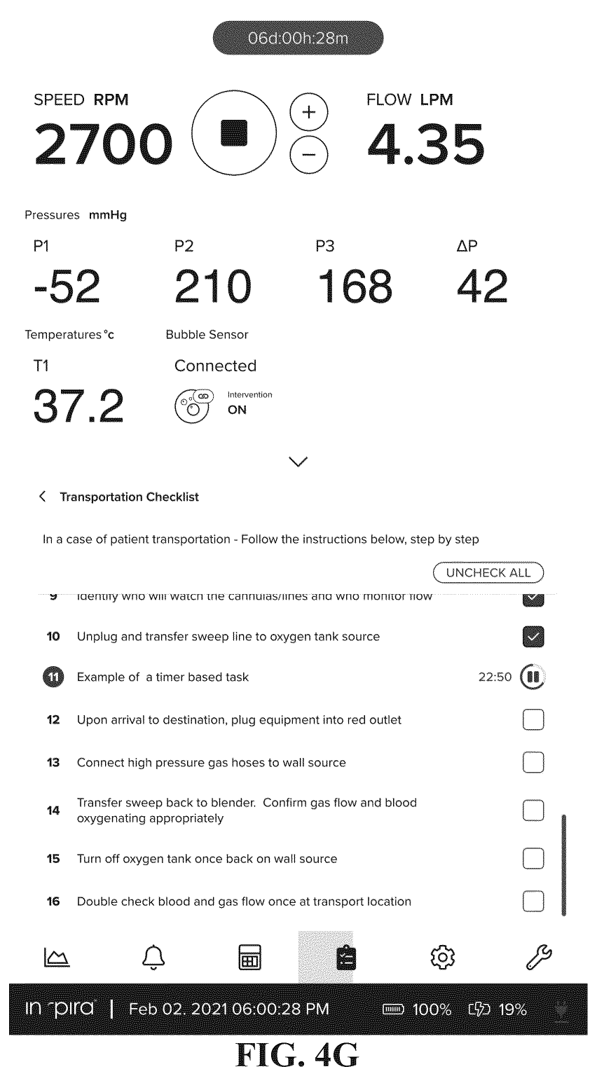

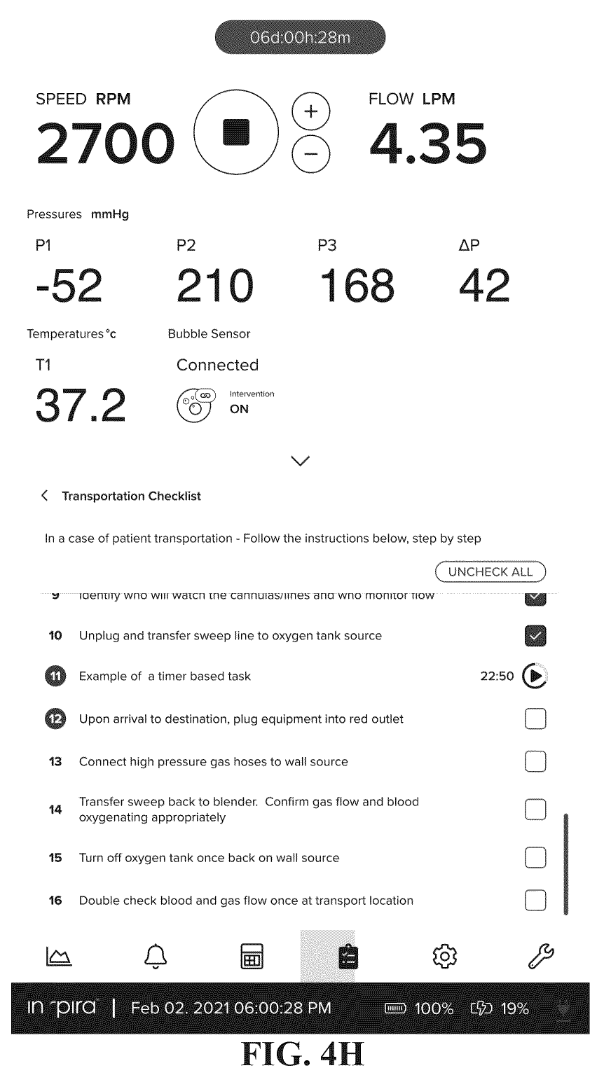

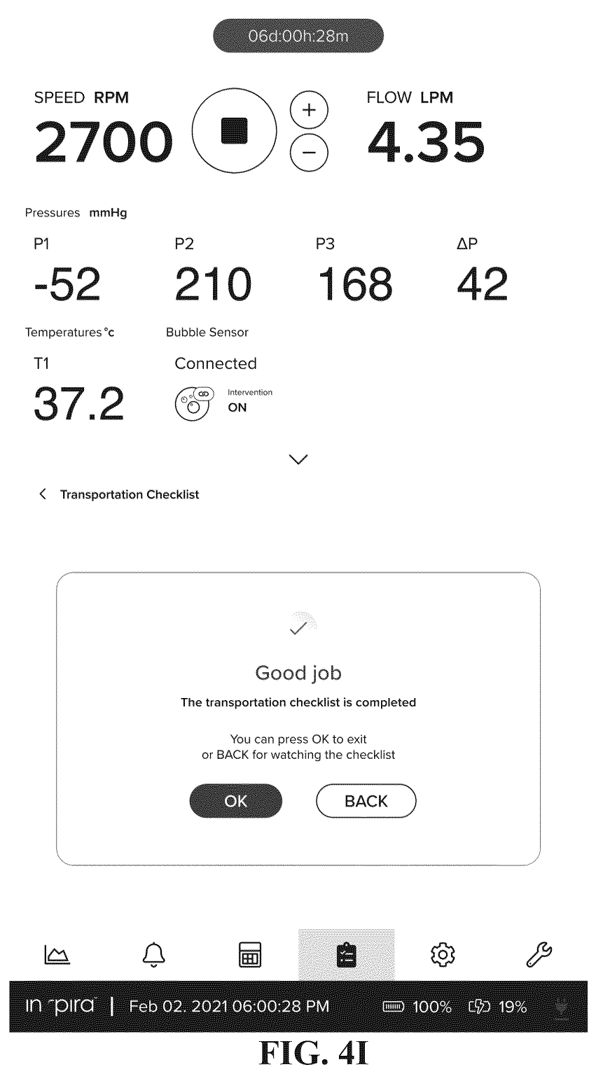

Reference is now made to FIGS. 4A-FIG. 4I which are illustrative screen shots of an exemplary interactive graphical user interface for automated monitoring of extracorporeal support patient care, according to some embodiments.

As shown on FIGS. 4A-FIG. 4I, the interactive graphical user interface (GUI) may be incorporated in a man-machine interface, e.g., a display and/or the like, of an extracorporeal support device, optionally integrated therewithin. For example, optionally in addition to various parameters of extracorporeal support device and/or vital signs of the patient supported, such as a rotational speed, i.e., revolutions per minute (RPM), flow rate, i.e., liters per minute (LPM), pump pressures and/or pressure differential, body temperature, and/or the like, there may be provided one or more components for allowing user interaction with functional modules of automated extracorporeal support patient care monitoring, such as described and illustrated herein.

FIG. 4A shows an exemplary screen shot of a menu of available operational modes and associated checklists for selection by a user. In the illustrated example on FIG. 4A, such operation modes may include, for example, checklists entitled “Patient Transportation” and “Weaning”, respectively. The selection of an operation mode may be performed, for example, by pressing a “Start” button provided via the interactive graphical user interface with relation to a respective operation mode, such as illustrated on FIG. 4A, and/or the like.

FIG. 4B shows another exemplary screenshot of the menu illustrated on FIG. 4A, with exemplary indication of user progress in a checklist associated with one of the operation modes. For example, as shown on FIG. 4B, a numerical indication of completed items out of a total number of items on the checklist may be provided, e.g., “9/16 steps” and/or the like. Additionally, or alternatively, a progress bar illustrating a percentage of completed items from the checklist may be displayed, e.g., as a partial fill out of a “Continue” button provided via the interactive graphical user interface with relation to the respective operation mode.

FIG. 4C shows an exemplary screen shot of a plurality of checklist items presented to a user for prompting confirmation of performance thereof. Optionally, the items may be sequentially ordered and presented to the user for confirmation in accordance with that order, such that the user may be prevented from moving on to confirming a next item until a current item has been confirmed. Optionally the user confirmation may be provided in form of checking a checkbox, such as illustrated on FIGS. 4C-4H herein, and/or the like. Optionally, the current item may be presented in a highlight and/or likewise indication, such as illustrated on FIG. 4C with relation to an item at a beginning of the presented checklist, denoted as item number “1”.

In some embodiments, the checklist may be a preloaded customized checklist entered by authorized personnel, e.g., an appointed administrator on behalf of a medical facility and/or institution, where the different check items may be phrased and ordered according to institution preference, reflecting its current best practice procedure, and/or the like. Additionally, or alternatively, the checklist items may be loaded from a default checklist provided with manufacture settings.

FIG. 4D shows another exemplary screen shot of the plurality of checklist items as illustrated on FIG. 4C, with the three foremost items on the checklist having been already confirmed, i.e., checked by a user, and a subsequent item, denoted as item number “4”, presented with a highlight. Optionally, as further illustrated on FIG. 4D, an indication of user progress on the checklist may be provided in a designated location on the display, for example, as a progress bar on a tab of functional modules for user selection, as shown at a bottom portion of the exemplary screen shots. Optionally the progress bar may be remained visible when other tabs being selected, thereby reminding the user that the checklist needs to be resumed and completed.

FIG. 4E shows yet another exemplary screen shot of the plurality of checklist items as illustrated on FIG. 4C-FIG. 4D, with all checklist items presented on the GUI having been confirmed, i.e., checked, by a user.

FIG. 4F shows an exemplary screen shot of a different plurality of checklist items, with all checklist items up to and including a tenth item denoted “10” having been checked, and an eleventh item currently being presented having a timer associated with it. The timer may be utilized by the user for providing reminder to move to a next item in the checklist after a predetermined time specified, e.g., 30 minutes as illustrated in the example shown on FIG. 4F.

Once the timer is expired, an indication may be presented to the user for drawing their attention to the checklist and/or item in question (not shown).

FIG. 4G shows another exemplary screen shot of the different plurality of checklist items as illustrated on FIG. 4F, with the timer after it had been set by a user and the predetermined time interval partially elapsed. FIG. 4H shows yet another exemplary screen shot of the different plurality of checklist items as illustrated on FIG. 4F, with the timer after the time interval partially elapsed. In some embodiments, the timer may optionally be paused and subsequently resumed by the user, such as illustrated on FIGS. 4G and 4H respectively.

FIG. 4H shows an exemplary screen shot of a notification presented to a user in response to completion of a checklist, in accordance with some embodiments. Optionally, a timing of starting and/or completing of a checklist and/or of checking items therein may be recorded for future review in a corresponding data store, such as a patient electronic medical record and/or the like, such as described and illustrated herein.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant extracorporeal support patient care tools and/or techniques will be developed, and the scope of the term “extracorporeal support” is intended to include all such new technologies a priori.

As used herein the term “about” refers to +10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment may include a plurality of “optional” features unless such features conflict.

Throughout this application, various embodiments may be presented in a range format. It should be understood that the description in range format is merely for convenience and brevity and should not be construed as an inflexible limitation on the scope of embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of embodiments, which are, for clarity, described in the context of separate embodiments, may also be provided in combination in a single embodiment. Conversely, various features of embodiments, which are, for brevity, described in the context of a single embodiment, may also be provided separately or in any suitable sub combination or as suitable in any other described embodiment. Certain features described in the context of various embodiments are not to be considered essential features of those embodiments, unless the embodiment is inoperative without those elements.

Although embodiments have been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A system for automated monitoring of extracorporeal support patient care, comprising:
a processing circuitry adapted to execute a code for:
providing an interactive user interface adapted to receive user selection of an operating mode for treatment of a patient supported with an extracorporeal support device;
retrieving from a database a checklist associated with the operating mode selected, the checklist comprising a plurality of action items required to be performed by a medical staff in treatment of the patient at a set of time intervals;
presenting via the interactive user interface the plurality of action items in accordance with the set of time intervals for prompting user confirmation of performing a respective portion of the plurality of action items presented at a respective timepoint; and
outputting an alert in response to not receiving user confirmation within a predetermined timeout.

2. The system of claim 1, wherein the interactive user interface is provided via an integrated man-machine interface of the extracorporeal support device.

3. The system of claim 1, wherein the checklist comprising at least one subset of the plurality of action items which members thereof are sequentially ordered, wherein the processing circuitry is further adapted to execute a code for presenting members of a respective one of the at least one subset in a respective sequential order.

4. The system of claim 3, wherein presenting a subsequent action item of a respective one of the at least one subset is contingent on receiving user confirmation of performing an action item presented.

5. The system of claim 1, wherein at least one of said plurality of action items is associated with a timer, wherein the processing circuitry is further adapted to execute a code for suspending of prompting user confirmation until expiry of the timer of the respective action item.

6. The system of claim 5, wherein the processing circuitry is further adapted to execute a code for outputting user notification in response to expiry of the timer.

7. The system of claim 1, wherein the processing circuitry is further adapted to execute a code for providing via the interactive user interface an indication of user progress on the checklist.

8. The system of claim 7, wherein the processing circuitry is further adapted to execute a code for overlaying the indication of user progress on an output provided via another user interface of the extracorporeal support device.

9. The system of claim 1, wherein the operating mode is selected from the group consisting of: medical staff shift change, supported patient transportation, patient support initiation, and supported patient weaning.

10. The system of claim 1, wherein the processing circuitry is further adapted to execute a code for recording information relating to user execution of the checklist in at least one data store.

11. The system of claim 10, wherein the at least one data store comprising an electronic medical record.

12. The system of claim 1, wherein the processing circuitry is further adapted to execute a code for loading the checklist from a user input received.

13. The system of claim 12, wherein the user input is provided in a tabular data format.

14. The system of claim 1, wherein the operating mode having a mode transition checklist associated therewith, the mode transition checklist comprising another plurality of action items on which switching to the operating mode is preconditioned, wherein the processing circuitry is further adapted to execute a code for: retrieving from the database the mode transition checklist, presenting via the interactive user interface the another plurality of action items for prompting user confirmation of performing thereof, and outputting an alert in response to not receiving user confirmation within a predetermined timeout.

15. A method for automated monitoring of extracorporeal support patient care, comprising:
providing an interactive user interface adapted to receive user selection of an operating mode for treatment of a patient supported with an extracorporeal support device;
retrieving from a database a checklist associated with the operating mode selected, the checklist comprising a plurality of action items required to be performed by a medical staff in treatment of the patient at a predetermined synchronized manner;
presenting via the interactive user interface the plurality of action items in accordance with the predetermined synchronized manner for prompting user confirmation of performing a respective portion of the plurality of action items presented at a respective timepoint; and
outputting an alert in response to not receiving user confirmation within a predetermined timeout.

16. A non-transitory computer readable medium storing a software program comprising data and computer implementable instructions for carrying out the method of claim 15.
